(19) United States
(12) Patent Application Publication
(10) Pub. No.: US 2026/0000385 A1
(43) Pub. Date: Jan. 1, 2026

(54) RELAY DEVICE AND ULTRASOUND DIAGNOSTIC APPARATUS

(71) Applicant: FUJIFILM Corporation, Tokyo (JP)

(72) Inventor: Takahide TERADA, Tokyo (JP)

(73) Assignee: FUJIFILM Corporation, Tokyo (JP)

(21) Appl. No.: 19/228,019

(22) Filed: Jun. 4, 2025

(30) Foreign Application Priority Data
Jun. 28, 2024 (JP) 2024-104988

(51) Int. Cl. A61B 8/00; A61B 8/12; B06B 1/02

(52) U.S. Cl. CPC A61B 8/4461; A61B 8/12; B06B 1/0215; B06B 2201/76

(57) ABSTRACT

A relay module is connected to a console via a cable and is connected to a transducer module in a body cavity insertion-type probe via a cable. The relay module includes a clock generation unit that generates a high frequency clock based on a low-frequency clock output from the console. The transducer module included in the probe executes signal processing for transmission beam forming based on a high frequency clock from the relay device.

### CROSS REFERENCE TO RELATED APPLICATION

This application claims priority to Japanese Patent Application No. 2024-104988 filed on Jun. 28, 2024, which is incorporated herein by reference in their entireties including the specifications, claims, drawings, and abstracts.

### BACKGROUND OF THE INVENTION

### 1. Field of the Invention

The present disclosure relates to a relay device and an ultrasound diagnostic apparatus including the relay device, and particularly to an ultrasound diagnostic apparatus comprising a body cavity insertion-type ultrasound probe.

### 2. Description of the Related Art

As a body cavity insertion-type ultrasound probe, a small-diameter probe such as an intravascular ultrasound (IVUS) probe or an ultrasound endoscope is known. The IVUS probe is also called a catheter-type ultrasound probe.

In an electronic scanning IVUS probe in the related art, an annular flexible printed circuit (FPC) substrate is disposed in a tip portion of the electronic scanning IVUS probe. An annular transducer array and an electronic circuit connected to the transducer array are provided on an inner side of the annular FPC substrate. Note that, JP2013-165865A discloses an ultrasound catheter device that performs phase control of a transmission signal to scan an ultrasound beam.

An IVUS ultrasound diagnostic apparatus generally has the above-described IVUS probe and an apparatus main body that displays an ultrasound image generated by controlling transmission and reception of ultrasound in the IVUS probe on a display device. The apparatus main body is also called a console.

A relay module is provided between the IVUS probe and the console as necessary. The relay module relays data exchanged between the IVUS probe and the console.

For example, JP2022-544560A describes an ultrasound imaging system comprising an imaging engine (console), a patient interface module (relay module), and an imaging assembly (IVUS probe). The patient interface module is connected to each of the console and the imaging assembly via a wire (cable). The patient interface module relays a command from the imaging engine to the imaging assembly. In addition, the patient interface module relays the ultrasound signal data and the like generated by the imaging assembly to the imaging engine.

### SUMMARY OF THE INVENTION

As described above, the electronic circuit is provided in the tip portion of the body cavity insertion-type probe. For example, a clock is required to execute signal processing for beam forming in the electronic circuit. A diameter of the tip portion of the probe is extremely small, and it is not practical to incorporate a clock generator into the electronic circuit.

It is also conceivable to generate a clock used for the electronic circuit in the ultrasound diagnostic apparatus main body and to supply the clock from the ultrasound diagnostic apparatus main body to the electronic circuit. However, in this case, various problems as described below may occur.

For example, in a case where the frequency of the clock is a high frequency, there may be a problem such as attenuation or distortion of a clock waveform in a long-distance transmission process from the apparatus main body to the electronic circuit. Alternatively, in a case where a plurality of types of clocks need to be supplied to the electronic circuit, there may be a problem that the configuration and control of the ultrasound diagnostic apparatus main body may be complicated. It should be noted that, although it is conceivable to transmit the clock according to a standard (for example, low voltage differential signal (LVDS)) that can perform data transmission at a high speed, in this case, there is a problem of an increase in size of the electronic circuit or an increase in power consumption in the electronic circuit.

An object of the present disclosure is to generate a clock used in a body cavity insertion-type probe between the body cavity insertion-type probe and an ultrasound diagnostic apparatus main body. Alternatively, an object of the present disclosure is to prevent or suppress an increase in the cable thickness of the body cavity insertion-type probe in a case where a plurality of types of clocks are supplied to an electronic circuit in the body cavity insertion-type probe.

A relay device according to the present disclosure comprises a first connection portion that is connected, via a first cable, to an ultrasound diagnostic apparatus main body having an original clock generation unit that generates an original clock, a second connection portion that is connected, via a second cable, to a probe that is inserted into a body cavity, and a probe clock generation unit that generates a probe clock used in the probe based on the original clock supplied from the ultrasound diagnostic apparatus main body via the first cable, in which the probe clock is supplied to the probe via the second cable.

In addition, the probe clock generation unit generates, as the probe clock, a first probe clock used in generating a delay data set used for beam forming in the probe. A frequency of the first probe clock may be higher than a frequency of the original clock.

Further, the probe clock generation unit may further generate, as the probe clock, a second probe clock used in processing a parameter set used for the beam forming in the probe, and a frequency of the second probe clock may be different from a frequency of the first probe clock.

Further, the second cable may have a signal line for a clock, the first probe clock may be output from the probe clock generation unit to the signal line for a clock in a first period, and the second probe clock may be output from the probe clock generation unit to the signal line for a clock in a second period different from the first period.

Further, the relay device may include a circuit that outputs, to the probe, a signal representing a type of the probe clock output to the signal line for a clock.

An ultrasound diagnostic apparatus according to the present disclosure comprises an ultrasound diagnostic apparatus main body having an original clock generation unit that generates an original clock, a probe that is inserted into a body cavity, and a relay device that is connected, via a first cable, to the ultrasound diagnostic apparatus main body and is connected, via a second cable, to the probe, the relay device including a probe clock generation unit that generates a probe clock used in the probe based on the original clock and outputting the probe clock to the probe via the second cable.

In addition, the probe clock generation unit may selectively generate, as the probe clock, a first probe clock having a first frequency and a second probe clock having a second frequency lower than the first frequency, the probe may have an electronic circuit that executes beam forming, and the electronic circuit may generate a delay data set used for the beam forming based on the first probe clock, and may store a parameter set used for the beam forming, which is a parameter set transmitted from the relay device, based on the second probe clock.

Further, the relay device may output the first probe clock and the second probe clock to the probe via a signal line for a clock in the second cable, and output a signal representing a type of the probe clock to the probe via the second cable, and the electronic circuit may identify a type of the probe clock input via the signal line for a clock according to the signal representing the type.

Further, the parameter set may include a first parameter set that is common over a plurality of times of the beam forming and a second parameter set that is changeable for each beam forming, the relay device may output the first parameter set and the second parameter set as the parameter set to the probe via the second cable, and may output a signal representing a storage location of the parameter set to the probe, and the electronic circuit may store the first parameter set in a first storage location and stores the second parameter set in a second storage location based on the signal representing the storage location of the parameter set.

Further, the second cable may have a first signal line, a second signal line, a third signal line, and a fourth signal line, the first signal line may be the signal line for a clock, the second signal line may be a signal line for transmitting the parameter set from the relay device to the electronic circuit and for transmitting a first reception signal from the electronic circuit to the relay device, the third signal line may be a signal line for transmitting the signal representing the storage location of the parameter set from the relay device to the electronic circuit and for transmitting a second reception signal from the electronic circuit to the relay device, and the fourth signal line may be a signal line for transmitting the signal representing the type of the probe clock from the relay device to the electronic circuit.

According to the present disclosure, the clock used in the body cavity insertion-type probe is generated between the body cavity insertion-type probe and the ultrasound diagnostic apparatus main body. Alternatively, according to the present disclosure, in a case where a plurality of types of clocks are supplied to an electronic circuit in the body cavity insertion-type probe, an increase in the cable thickness of the body cavity insertion-type probe can be prevented or suppressed.

### BRIEF DESCRIPTION OF THE DRAWINGS

FIG. 1 is a block diagram showing a schematic configuration of an ultrasound diagnostic apparatus according to Embodiment 1.

FIG. 2 is a block diagram showing an example of a configuration of a transducer module according to Embodiment 1.

FIG. 3 is a diagram showing a plurality of signals (including a clock) to be relayed by a relay module in Embodiment 1.

FIG. 4 is a timing chart showing various signals exchanged between the relay module and the transducer module in a case where transmission beam forming is executed in Embodiment 1.

FIG. 5 is a block diagram showing an example of a configuration of a body cavity insertion-type probe according to Embodiment 2.

FIG. 6 is a timing chart showing various signals exchanged between a relay module and a transducer module in a case where ultrasound diagnosis is performed in Embodiment 2.

### DESCRIPTION OF THE PREFERRED EMBODIMENTS

Hereinafter, embodiments according to the present disclosure will be described with reference to the drawings.

### Embodiment 1

### Configuration of Ultrasound Diagnostic Apparatus

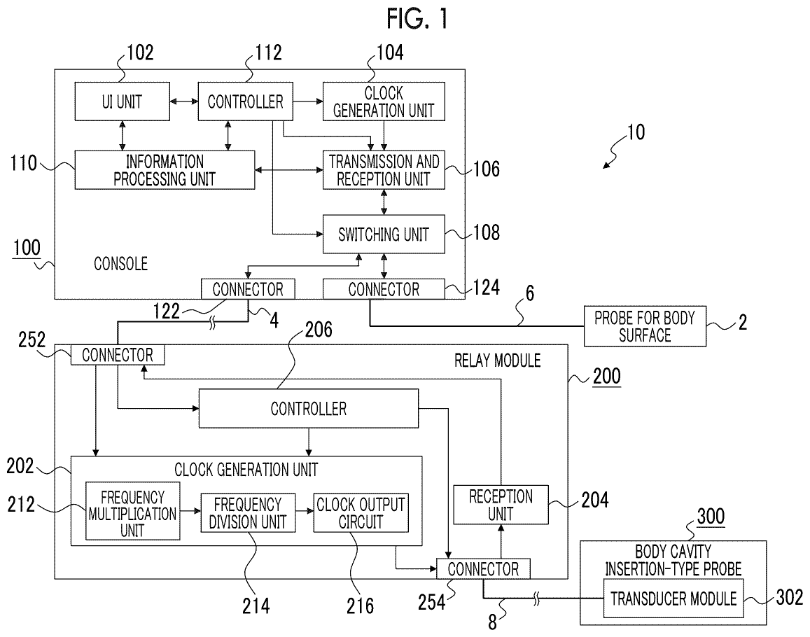

FIG. 1 is a block diagram showing a schematic configuration of an ultrasound diagnostic apparatus in the present embodiment. An ultrasound diagnostic apparatus 10 in the present embodiment has an apparatus main body (hereinafter, also referred to as a “console”) 100, a relay module 200, and probes 2 and 300. The ultrasound diagnostic apparatus 10 can also be referred to as an ultrasound diagnostic system.

In the present embodiment, two types of probes, that is, a probe 2 for a body surface and a body cavity insertion-type probe 300, are connected to the console 100. Only the probe 300 may be connected to the console 100, or another probe may be further connected to the console 100.

The probe 2 shown in the drawing is a probe that is in contact with a surface of a subject and performs ultrasound diagnosis in that state (for example, a linear probe or a convex probe).

The probe 300 is a body cavity insertion-type ultrasound probe. Specifically, the probe 300 is an electronic scanning IVUS probe that is inserted into a blood vessel of a subject. The probe 300 is a long member having flexibility. An outer diameter of the probe 300 is, for example, in a range of 1 to 3 mm. The outer diameter of the probe 300 may be in the range thereof or less, or in the range thereof or more. The probe 300 has a hollow passage formed along a central axis thereof. The guide wire is inserted into the passage. The probe 300 advances in the blood vessel along the guide wire already disposed in the blood vessel of the subject.

The ultrasound diagnostic apparatus 10 in the present embodiment has a function of supporting a user who performs ultrasound diagnosis using beam forming. In the present embodiment, in a case where the term “beam forming” is simply used, both transmission beam forming and reception beam forming are included.

The console 100 and the relay module 200 are connected by a cable 4 as a first cable. Specifically, one end of the cable 4 is connected to a connector 122 of the console 100. In addition, the other end of the cable 4 is connected to a connector 252 that is a first connection portion of the relay module 200. The probe 2 is connected to the console 100 by the cable 6. Specifically, one end of the cable 6 is connected to a connector 124 of the console 100. The other end of the cable 6 is connected to the probe 2. The probe 300 is connected to the relay module 200 by a cable 8 as the second cable. One end of the cable 8 is connected to a connector 254 that is a second connection portion of the relay module 200. The other end of the cable 8 is connected to the probe 300.

A transducer module 302 is disposed in the tip portion of the probe 300 and includes an electronic circuit. In FIG. 1, the cable 8 is shown as a configuration different from the probe 300. The cable 8 may also be a component included in the probe 300, as in the transducer module 302. In practice, since the cable 8 is a component that passes through the catheter, it is positioned as a component of the probe 300 in a broad sense.

The console 100 includes a user interface (UI) unit 102, a clock generation unit 104, a transmission and reception unit 106, a switching unit 108, an information processing unit 110, and a controller 112.

The user interface unit 102 includes a display section and an input section. The display section is configured with an organic EL display device, a liquid crystal display (LCD), or the like, and an ultrasound image or the like is displayed thereon. The input section is configured with an operation panel, an operation button, a keyboard, or the like operated by a user such as a technician. It should be noted that the display section and the input section may be configured with a touch screen panel or the like.

The clock generation unit 104 functions as an original clock generation unit. The clock generation unit 104 generates a clock to be transmitted to the probe 300 as an original clock in response to an instruction from the controller 112. The clock generation unit 104 generates, for example, a clock having a frequency of 20 MHz as an original clock. It should be noted that the clock generation unit 104 may generate a basic clock used in the console 100. In this case, the original clock may be generated from the basic clock. The original clock may be transmitted to the probe 2.

The transmission and reception unit 106 functions as a transmission circuit (transmission beam former) and a reception circuit (reception beam former) under the control of the controller 112. However, the transmission and reception unit 106 functions according to the specifications of the probes 2 and 300 in operation. For example, in a case where a plurality of transmission signals are generated in the probe for the transmission beam forming, the transmission and reception unit 106 basically does not function at the time of transmission. In a case where the sub reception beam forming is executed in the probe, the transmission and reception unit 106 executes the main reception beam forming.

The switching unit 108 selects any probe that is electrically connected to the transmission and reception unit 106 among the connected probes 2 and 300 under the control of the controller 112. The switching unit 108 may be configured with an electronic switch including a plurality of transistors or may be configured with a mechanical switch including a plurality of relays.

The information processing unit 110 executes various types of information processing in response to a user operation or the like. Specifically, the information processing unit 110 functions as a beam data processing unit, an image forming unit, a display processing unit, and the like. The information processing unit 110 generates an ultrasound image such as a tomographic image and displays the generated ultrasound image on the user interface unit 102. More specifically, the information processing unit 110 generates an ultrasound image based on the reception information (specifically, the reception frame data as the reception beam data string) and displays the ultrasound image. The controller 112 controls the operations of the other components 102 to 110.

The relay module 200 functions as a relay device that transmits data and the like from the console 100 to the probe 300 and transmits data and the like from the probe 300 to the console 100. The relay module 200 is generally installed in the vicinity of a bed on which the subject is placed. A length of the cable 8 that connects the relay module 200 and the transducer module 302 in the tip portion of the probe 300 is, for example, 1 to 2 m. On the other hand, a length of the cable 4 that connects the relay module 200 and the console 100 is, for example, 4 to 5 m.

The relay module 200 includes a clock generation unit 202, a reception unit 204, and a controller 206. The components not used in the description of the present embodiment are omitted from the drawings.

The clock generation unit 202 functions as a probe clock generation unit that generates a clock used in the probe 300 (hereinafter, also referred to as a “probe clock”) based on an original clock supplied from the console 100 via the cable 4 under the control of the controller 206.

The clock generation unit 202 in the present embodiment generates a first probe clock and a second probe clock as the probe clock. The first probe clock is a clock used in generating a delay data set (specifically, a plurality of transmission delay times) used for the transmission beam forming in the probe 300. A frequency of the first probe clock is higher than a frequency of the original clock.

In the present embodiment, for example, 20 MHz is used as the frequency of the original clock. In order to effectively perform the transmission beam forming, the first probe clock needs to have a frequency that is several times to a dozen times the center frequency of the ultrasound pulse (in a case of IVUS, for example, about 10 to 40 MHz). It should be noted, in order to obtain a high-quality ultrasound image by executing the transmission beam forming, it is preferable that the frequency of the first probe clock is set to about 100 to several hundred MHz. In the present embodiment, for example, a clock of 200 MHz is used as the first probe clock.

In the following description, since the frequency of the original clock is lower than the frequency of the first probe clock, the original clock may be referred to as a “low frequency clock” for convenience. In addition, since the frequency of the first probe clock is higher than the frequency of the original clock, and it is assumed that a frequency of about 100 to several hundred MHz is used as the frequency of the first probe clock, the first probe clock is sometimes referred to as a “high frequency clock” for convenience.

On the other hand, the second probe clock is a clock used in processing a parameter set used for the beam forming in the transducer module 302 (more specifically, the electronic circuit) in the probe 300. The processing of the parameter set includes processing of storing the parameter set in a register in the electronic circuit. More specifically, the register is configured with a plurality of storage elements connected in series. Serial data representing the parameter set is sequentially transmitted in accordance with a second probe clock, and the serial data is temporarily stored in the plurality of storage elements. The frequency of the second probe clock is different from the frequency of the first probe clock. Although details will be described later, in the present embodiment, the frequency of the second probe clock is lower than the frequency of the first probe clock.

The clock generation unit 202 includes a frequency multiplication unit 212, a frequency division unit 214, and a clock output circuit 216. The frequency multiplication unit 212 may be formed of, for example, a phase locked loop (PLL). The frequency multiplication unit 212 generates a clock having a frequency that is n times the frequency of the original clock based on the original clock transmitted from the console 100. Here, n is usually an integer of 2 or more, and is, for example, a numerical value in a range of 2 to 500. n is a fixed value, but n may be a variable value. The frequency division unit 214 selectively generates the frequency of the first probe clock and the frequency of the second probe clock by dividing the clock output from the frequency multiplication unit 212. Since the center frequency of the ultrasound pulse varies depending on the type of the probe, the frequency division unit 214 sets the number of times of frequency division of the first probe clock according to the type of the probe and the like, under the control of the controller 206. The frequency of the second probe clock may be set to be variable. The clock output circuit 216 performs output control of the clock generated by the frequency division unit 214. Specifically, the clock output circuit 216 outputs a probe clock or stops the output (for example, fixes the level of the signal to Low).

The reception unit 204 has a function of amplifying a reception signal input from the probe 300 via the cable 8. Specifically, the reception unit 204 includes two amplifiers that amplify two reception signals input in parallel. Each amplified reception signal is output to the console 100 via the cable 4. One reception signal may be transmitted for each transmission and reception between the transducer module 302 and the console 100. In this case, one amplifier is provided.

The controller 206 controls the operation of each component in the relay module 200. In particular, the controller 206 has a function of controlling the execution of beam forming in the probe 300 under the control of the controller 112. For example, the controller 206 generates a parameter set used for beam forming in response to an instruction from the console 100, and controls the generation of a clock of a predetermined frequency. In addition, the controller 206 performs input and output control of a plurality of signals via a plurality of signal lines included in the cable 8. For example, the controller 206 controls the probe clock generated by the clock generation unit 202 to be output to the probe 300 via a signal line for a clock output included in the cable 8.

It should be noted that the frequency multiplication unit 212 may be realized by a circuit different from the PLL. In this case, the clock generation unit 202 may convert the original clock into a probe clock having any frequency instead of multiplying the original clock.

### Configuration of Transducer Module 302

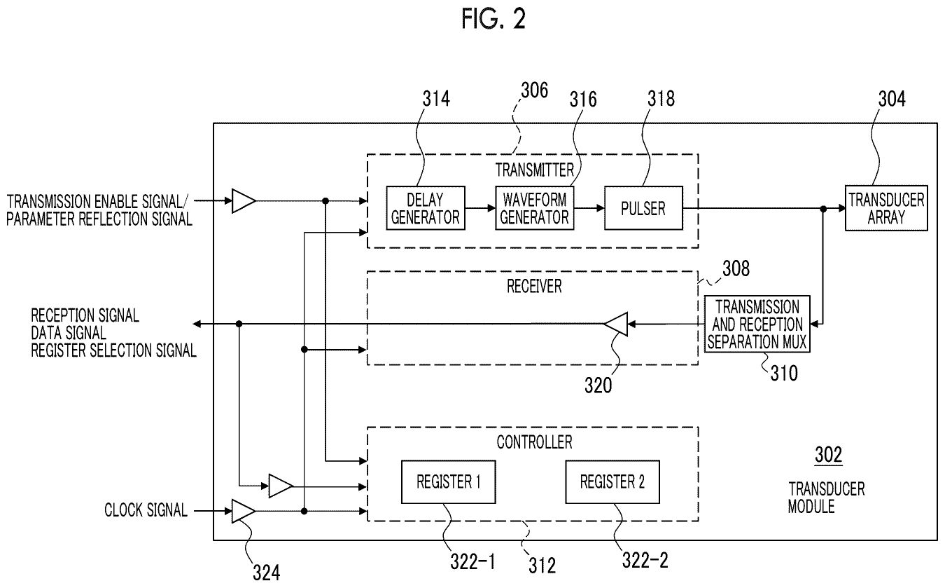

FIG. 2 is a diagram showing an example of a configuration of a transducer module 302 included in the probe 300 in the present embodiment. The transducer module 302 is a module provided in the tip portion of the probe 300, and has an electronic circuit and a transducer array connected thereto. The transducer module 302 has a configuration in which beam forming can be executed. In particular, the transducer module 302 in the present embodiment can perform signal processing for transmission beam forming based on a clock of a high frequency (about 100 to several hundred MHz).

The transducer module 302 in the present embodiment includes a transducer array 304, a transmitter 306, a receiver 308, a transmission and reception separation multiplexer (MUX) 310, and a controller 312. The transducer array 304 is provided at a tip of the transducer module 302 and is configured with, for example, a plurality of transducers arranged in a circular shape. The transmitter 306, the receiver 308, the transmission and reception separation multiplexer 310, and the controller 312 are an electronic circuit consisting of one or a plurality of integrated circuits (ICs) as a whole.

The transmitter 306 is a circuit for transmitting an ultrasound from the transducer array 304. The transmitter 306 executes processing for transmission beam forming based on the input clock under the control of the controller 312. In FIG. 2, as a configuration of the transmitter 306, one delay generator 314, one waveform generator 316, and one pulser 318 are shown. In practice, the delay generator 314, the waveform generator 316, and the pulser 318 are provided for each transducer constituting the transducer array 304. For example, in a case where the transducer array 304 is configured with 64 transducers, 64 delay generators 314, 64 waveform generators 316, and 64 pulsers 318 are provided. It should be noted that a multiplexer may be provided between the transmitter 306 and the transducer array 304. In this case, the transmission and reception separation multiplexer 310 may be caused to function in both the transmission process and the reception process.

The plurality of delay generators 314 generate a plurality of delay amounts (a plurality of delay times) corresponding to the plurality of transducers constituting the transmission opening based on the input clock (first probe clock). The plurality of delay amounts are also referred to as a transmission delay amount profile or a transmission delay data set. In general, one period of the clock defines the minimum delay amount. Each of the plurality of waveform generators 316 generates a transmission pulse having a predetermined waveform. Specifically, the plurality of waveform generators 316 generate a plurality of transmission pulses based on a plurality of delay amounts. The plurality of pulsers 318 generate the plurality of transmission pulses (a plurality of transmission signals) having a high voltage based on the plurality of transmission pulses generated by the plurality of waveform generators 316. The plurality of transmission pulses are output to a plurality of transducers constituting the transmission opening.

The plurality of transmission signals are supplied to the plurality of transducers, and a transmission beam is formed. That is, the ultrasound is radiated from the transducer array 304 to the inside of the living body. The reflected wave reflected by the biological tissue is received by the transducer array 304. As a result, reception signal strings are output from the transducer array 304. The transmission and reception separation multiplexer 310 has the same function as a transmission and reception separation switch of a general ultrasound diagnostic apparatus, and separates the high-voltage transmission signal output from the transmitter 306 and input to the transducer array 304 so that the transmission signal is not input to the receiver 308 that operates at a low voltage. The transmission and reception separation multiplexer 310 has a function as a multiplexer of the reception signal strings in addition to this. In the embodiment, the transmission and reception separation multiplexer 310 selects two reception signals that are spatially adjacent to each other for each reception. A single reception signal may be selected, or three or more reception signals may be selected.

The receiver 308 is a circuit that processes two reception signals output from the transmission and reception separation multiplexer 310. In the embodiment, the receiver 308 includes two amplifiers 320 that amplify two reception signals. However, in FIG. 2, only one amplifier 320 is shown for simplicity. The two amplified reception signals are transmitted to the console 100 via the relay module 200. In the reception circuit (reception beam former) in the console 100, a plurality of reception signals that are spatially arranged (for example, 16 reception signals) are subjected to delay processing and then added, thereby forming reception beam data. The reception dynamic focus technique is used in the delay processing. As will be described later, a plurality of reception signals may be phase-added in the transducer module 302.

The controller 312 controls the operations of the transmitter 306 and the receiver 308. The controller 312 in the present embodiment has two types of registers 322-1 and 322-2 as a storage section of a parameter set used in the transmission and reception processing. The controller 312 has a function of dividing and storing the parameter set transmitted from the relay module 200 in the register 322-1 or the register 322-2 according to the properties of the parameters. In the present embodiment, two separated parameter sets are transmitted from the relay module 200.

In a case where it is not necessary to distinguish between the registers 322-1 and 322-2, the registers 322-1 and 322-2 are collectively referred to as a “register 322”. The parameter set includes one or a plurality of parameters. In the present embodiment, the term “parameter” may indicate a type of a parameter or may indicate a set value of each parameter.

As described above, the parameter set used in the beam forming is stored in the register 322. The parameter set in the present embodiment includes a common first parameter set over a plurality of times of transmission and reception and a second parameter set changed for each transmission and reception. That is, the first parameter set consists of a plurality of parameters that are not changed for each transmission and reception. The second parameter set consists of a plurality of parameters that can be changed for each transmission and reception. For example, one register 322-1 (hereinafter, referred to as a “register 1”) functions as a first storage unit that stores the first parameter set. The other register 322-2 (hereinafter, referred to as a “register 2”) functions as a second storage unit that stores the second parameter set. In the following description, the first parameter set will be referred to as a “static parameter”, and the second parameter set will be referred to as a “dynamic parameter”. It should be noted that one transmission and reception are configured with one transmission and one reception following the transmission. The transmission and reception are repeated while the transmission opening and the reception opening are subjected to rotational scanning.

As shown in FIG. 2, various signals input to and output from the transducer module 302 include a transmission enable signal, two reception signals, a data signal, a register selection signal, and a clock signal. As described with reference to FIG. 1, the relay module 200 and the transducer module 302 are connected to each other via the cable 8. The cable 8 includes a plurality of signal lines for exchanging the various signals. The various signals will be described later.

In FIG. 2, an amplifier 324 is provided on a wiring line in the transducer module 302. Electronic components such as the amplifier 324 are provided as necessary. The electronic components may be comprised in the same IC as the transmitter 306, the receiver 308, or the like.

### Type of Signal Transmitted and Received by Relay Module 200

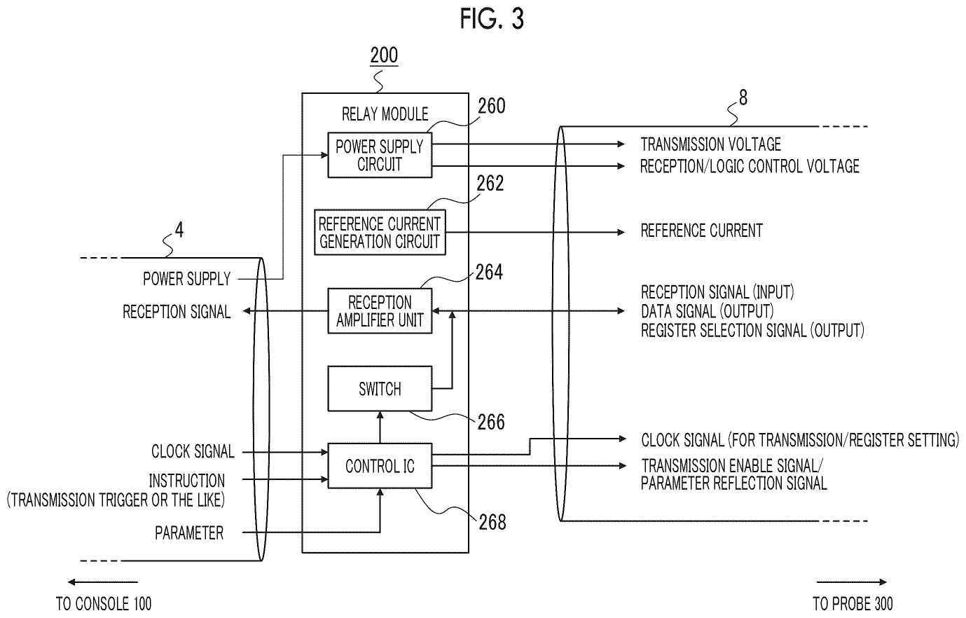

FIG. 3 is a diagram collectively showing signals and the like exchanged between the relay module 200 and the console 100 and between the relay module 200 and the probe 300 in the present embodiment. Further, FIG. 3 shows a hardware configuration that is not shown in FIG. 1. In FIG. 3, the relay module 200 includes a power supply circuit 260, a reference current generation circuit 262, a reception amplifier unit 264, a switch 266, and a control IC 268. The control IC 268 shown in FIG. 3 corresponds to the controller and the clock generator shown in FIG. 1. The reception amplifier unit 264 shown in FIG. 3 corresponds to the function as the reception circuit of the transmission and reception unit 106 shown in FIG. 1.

The power supply circuit 260 supplies power to the probe 300 for use therein. Among the power to be supplied, the transmission voltage is supplied to the transmitter 306. The reception/logic control voltage is supplied to the receiver 308 and the controller 312. The reference current generation circuit 262 supplies a reference current as a current signal for determining a bias current flowing in an analog circuit such as a reception amplifier. In the embodiment, the reception amplifier unit 264 is configured with two reception amplifiers arranged in parallel. Each reception amplifier amplifies each reception signal output from the probe 300.

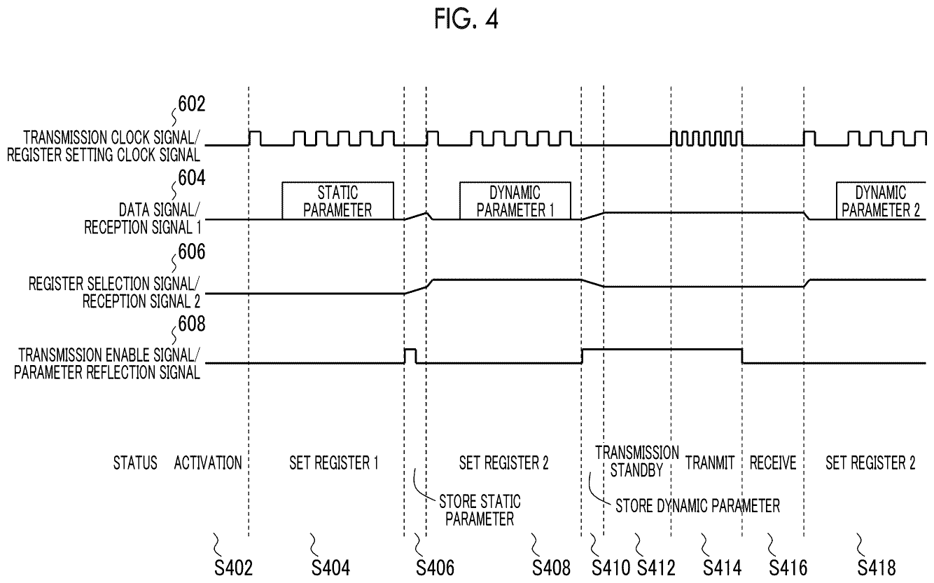

The switch 266 switches whether the signal lines (signal lines 604 and 606 shown in FIG. 4 below) are used for output (that is, for a data signal or a register selection signal) or are used for input (that is, for a reception signal) under the control of the control IC 268. The switch 266 may be configured with a three-state buffer. In a case of being configured with the three-state buffer, the control IC 268 controls the switch 266 to output the data signal or the value of the register selection signal and to be in a High Z (Open) state during reception of the reception signal. In a case where the state is set to High Z (Open), the data signal and the register selection signal are not output. As described above, by increasing the output impedance, it is possible to prevent noise from being mixed into the reception signal.

The control IC 268 performs control for transmitting a control signal and a parameter set to the probe 300 in response to an instruction from the console 100. In addition, the control IC 268 selectively generates the first probe clock and the second probe clock based on the original clock. The control IC 268 is configured with, for example, a field programmable gate array (FPGA).

### Configuration of Signal Line of Cable 8 Connecting Transducer Module 302

FIG. 4 is a timing chart of various signals exchanged between the relay module 200 and the transducer module 302 in a case where the transmission beam forming is executed in the present embodiment. In FIG. 4, the signal lines 602 to 608 included in the cable 8 are shown in association with various signals exchanged using the signal lines 602 to 608. The application of the various signals and the timing of transmitting and receiving the various signals will be described in detail later, and here, the types of signals and the states of the signals (that is, the states indicated by the levels of the signals (High or Low)) using the respective signal lines 602 to 608 will be briefly described. It should be noted that, for the level of the signal, High and Low may be used in reverse depending on the type or combination of the signal.

First, the signal line 602 is the signal line for the clock output (a first signal line) for transmitting the clock signal. Specifically, a transmission clock signal (hereinafter, also referred to as a “transmission clock”) used in generating the transmission delay time is transmitted as a first probe clock to the signal line 602. In addition, a register setting clock signal (hereinafter, referred to as a “register setting clock”) used in processing a parameter set used for the transmission beam forming is transmitted as a second probe clock to the signal line 602. That is, in the present embodiment, one signal line 602 is used as a signal line for transmission clock and a signal line for register setting clock without separately providing a clock signal line for register setting.

The signal line 604 is a second signal line for transmitting a data signal. The data signal in the present embodiment is a signal representing a parameter set used in beam forming or the like. In addition, the signal line 604 is also a signal line for transmitting the first reception signal from the transducer module 302 to the relay module 200. That is, the signal line 604 is used as a signal line for transmitting the data signal and a signal line for transmitting the first reception signal. The signal line 606 is a third signal line that transmits a register selection signal representing a storage location of the parameter set. The register selection signal can also be said to be a signal for distinguishing between a first period in which the static parameter is output to the register 1 and a second period in which the dynamic parameter is output to the register 2. As a matter of course, the second period is a period different from the first period. In the present embodiment, in a case where the static parameter set in the register 1 is output, the level of the register selection signal is set to Low. On the other hand, in a case where the dynamic parameter set in the register 2 is output, the level of the register selection signal is set to High. In addition, the signal line 606 is also used as a signal line for transmitting the second reception signal from the transducer module 302 to the relay module 200.

In the present embodiment, two reception signals (the first reception signal and the second reception signal) output from the receiver 308 are transmitted by using two signal lines of the signal line 604 and the signal line 606. It should be noted that the transmission efficiency can be improved as the number of signal lines for transmitting the reception signal is increased. Therefore, the number of signal lines in the cable 8 may be increased (three or more, for example, four signal lines may be used for the reception signal transmission). This prioritizes transmission efficiency over the reduction in cable diameter.

The signal line 608 is provided as a fourth signal line for transmitting a control signal representing the types of the transmission clock (first probe clock) and the register setting clock (second probe clock). In the present embodiment, the type of the probe clock is identified by the High/Low of the level of the signal. For example, a period in which the level of the control signal is in a state of High is an output period of the transmission clock (or a transmission standby period). That is, the period is a transmission enable period (a period in which transmission is possible). From that viewpoint, the control signal is a transmission enable signal. On the other hand, a period in which the level of the control signal is in a state of Low is a period in which the transmission enable period is not set, that is, a period in which the transmission is not possible, and is a setting period of the parameter. It should be noted that the period in which the level of the control signal is in the state of Low is also a period in which the receiver 308 can be operated.

In a case where the level of the control signal on the signal line 608 is changed from Low to High, the controller 312 determines that the storage of the parameter set in the register 322 is ended, and transmits the parameter set from the register 322 to another circuit (including the transmitter 306 and the receiver 308). From that viewpoint, the control signal is a parameter reflection signal for instructing the reflection (specifically, the transmit) of the parameter set. Specifically, after the storage of the static parameter set in the register 1 is ended, a single pulse is generated as a part of the control signal. The single pulse corresponds to the parameter reflection signal. The static parameter set is transmitted from the register 1 to the other circuit at a time point of a rising edge (change from Low to High) in the single pulse. After the end of the storage of the dynamic parameter set in the register 2, a rising edge occurs in the control signal. At that time, the dynamic parameter set is transmitted from the register 2 to the other circuit. It should be noted that the single pulse is generated before the first transmission and reception in a transmission and reception sequence consisting of a series of a plurality of transmissions and receptions.

An electronic component such as a three-state buffer may be provided on the wiring line in the transducer module 302 shown in FIG. 2, as necessary. The electronic components may be comprised in the same IC as the transmitter 306, the receiver 308, or the like. The electronic component may selectively switch a destination of signal transmission or electrically disconnect another signal line from a specific signal line.

For example, in a case where the transmission enable signal is output to the signal line 608 (in a case where the level of the signal is High), the signal line 602 and the controller 312 may be electrically disconnected from each other such that the clock signal (transmission clock) input from the signal line 602 is output only to the transmitter 306. On the other hand, in a case where the transmission enable signal is not output to the signal line 608 (in a case where the level of the signal is Low), the signal line 602 and the transmitter 306 may be electrically disconnected from each other such that the clock signal (register setting clock) input from the signal line 602 is output only to the controller 312.

### Execution Control of Transmission Beam Forming

Next, the execution control of the transmission beam forming executed in the present embodiment will be described while clarifying the states of various signals transmitted through the signal lines 602 to 608 shown in FIG. 4. It should be noted that the switching unit 108 selects the probe 300 as a data exchange partner in response to the instruction from the controller 112.

The relay module 200 receives the original clock from the console 100 until the selection is canceled (the connection is canceled) in a case where the probe 300 is selected on the console 100 by the user operation (or in a case where the connection of the probe 300 is automatically detected).

A user (a doctor, an examination technician, or the like) operates the console 100 to give various instructions prior to the examination or imaging using ultrasound (and during the examination or imaging using ultrasound). For example, the user may give an instruction as to whether the displayed image has high image quality or low image quality. Alternatively, the user instructs the setting (or change) of the frame rate, the imaging mode (B-mode, blood flow, or the like), various measurement functions (the area of the blood vessel lumen, the blood vessel diameter, the stenosis rate, or the like), the imaging range, the transmission focal condition (for example, the focal depth (focus distance)), and the like. Further, the user instructs the clock, the transmission and reception sequence, and the setting (or change) of the signal processing or the image processing on the console 100.

The controller 206 of the relay module 200 in the present embodiment generates a parameter set in response to these instructions. The relay module 200 generates a static parameter set at the beginning of the transmission and reception sequence, that is, prior to the first transmission and reception, and transmits the static parameter set to the probe 300. The relay module 200 generates a dynamic parameter set prior to for each transmission and reception, and transmits the dynamic parameter set to the probe 300.

The controller 206 determines the frequency to be generated with reference to the instruction from the user. For example, in a case where the controller 206 determines that a high-accuracy examination or the like is required, the controller 206 instructs the clock generation unit 202 to generate a clock having a frequency of, for example, 200 MHz. Further, in a case where the controller 206 determines that the medium-accuracy examination or the like is required, the controller 206 instructs the clock generation unit 202 to generate a clock having a frequency of, for example, 160 MHz. Furthermore, in a case where the controller 206 determines that the low-accuracy examination or the like is sufficient, the controller 206 instructs the clock generation unit 202 to generate a clock having a frequency of, for example, 100 MHz. In a case where a clock of a frequency of 160 MHz is generated in a case where the center frequency of the transmission pulse is 20 MHz, the controller 206 sets a multiplication number to 8. In this case, the frequency multiplication unit 212 outputs a clock of a frequency of 20×8=160 MHz.

In addition, the controller 112 calculates a transmission focus distance (a profile of a transmission delay amount) suitable for the diagnosis depth instructed by the user. Incidentally, the maximum value of the transmission delay amount is determined by the clock frequency and the number of stages of the delay line constituting the delay generator 314. For example, in a case where the clock frequency is 200 MHz and the number of stages of the delay line is 16, the maximum value of the transmission delay amount is 200 MHz (5 ns)×16=80 ns.

The controller 206 according to the embodiment has a function of calculating a delay resolution required for the transmission beam forming based on the result of calculating the transmission delay profile and the center frequency of the transmission pulse. In a case where the delay resolution is low, the accuracy of the transmission beam forming is lowered, and the image quality is deteriorated. On the other hand, even in a case where the delay resolution is excessively high, the accuracy of the transmission beam forming is saturated and the effect is not obtained. Therefore, for example, in a case where the center frequency of the transmission pulse is doubled, the delay resolution is doubled.

Specifically, the controller 206 determines the frequency of the clock used for the transmission beam forming based on the maximum value of the transmission delay amount and the delay resolution. As a result, the controller 206 suppresses power consumption by not generating a clock having a frequency higher than necessary.

In addition, the controller 206 sets the reception gain in the transducer module 302 or the reception gain in the relay device to an optimum value according to the reception gain setting value.

As described above, the controller 206 generates a parameter set to be transmitted to the probe 300. The dynamic parameter set includes a plurality of parameters that define a transmission delay amount, a transmission opening, a reception opening, and the like. On the other hand, the static parameter set includes a plurality of parameters for defining the transmission pulse waveform, the transmission frequency, the reception gain, time gain compensation (TGC), and the like. Of course, each parameter set may include parameters other than the above-described parameters. Hereinafter, processing of setting the parameter set in the register 322 in the transducer module 302 will be described in more detail.

The timing chart shown in FIG. 4 schematically shows a temporal change in the status. For example, a time length of each status period is a simple example, and the change in each signal is also a simple example. In FIG. 4, a horizontal axis is a time axis.

In S402, at a point in time when the relay module 200 is activated, the level of the signal on each of the signal lines 602 to 608 is the initial level, that is, Low.

In S404 after the activation, the static parameter set transmitted from the relay module 200 is stored in the register 1. Specifically, the relay module 200 outputs a data signal representing the static parameter set through the signal line 604, and maintains a level of the register selection signal output through the signal line 606 to Low. Low indicates that the parameter set storage destination is the register 1. Further, the relay module 200 outputs a register setting clock signal (second probe clock) through the signal line 602. In the example shown in the drawing, a single pulse indicating the start of the register setting status is output prior to the register setting clock signal.

Subsequently, in a case where the storage of the static parameter set in the register 1 is completed, the relay module 200 sets the level of the control signal on the signal line 608 to High for a short time, that is, generates a single pulse. As a result, the end of the parameter setting period is notified to the controller 312. Due to the change in the signal level on the signal line 608, the controller 312 in the transducer module 302 transmits the static parameter set temporarily stored in the register 1 to another circuit to construct a status in which the static parameter set can be actually used.

In S406, the relay module 200 transitions the level of the register selection signal on the signal line 606 from Low to High in order to prepare for the next S408.

In S408, the dynamic parameter set transmitted from the relay module 200 is stored in the register 2. Specifically, the relay module 200 outputs a data signal representing the dynamic parameter set through the signal line 604. The level of the register selection signal output through the signal line 606 is High. High indicates that the parameter set storage destination is the register 2. In addition, the relay module 200 outputs a register setting clock signal (second probe clock) through the signal line 602.

In a case where the storage of the dynamic parameter set in the register 2 is completed, in S410, the relay module 200 sets the level of the control signal on the signal line 608 to High. The high in S410 corresponds to a single pulse corresponding to the parameter reflection signal. A change from Low to High in the control signal means an end of the parameter setting period. Based on the change, the controller 312 in the transducer module 302 transmits the dynamic parameter set stored in the register 2 to another circuit to construct a status in which the dynamic parameter set can be actually used. In S412, the level of the signal is maintained at High, but High indicates a transmission enable state. The control signal having a High level corresponds to the transmission enable signal.

As described above, the parameter set used in the transmission beam forming is stored in the transducer module 302, so that the transmission beam forming can be executed.

In addition, in a case where the level of the control signal on the signal line 608 becomes High, the transmission of the dynamic parameter set stored in the register 2 is started as described above, but at the same time, the transmitter 306 and the receiver 308 start the operation. In this case, the relay module 200 opens the switch 266 shown in FIG. 3 to disconnect the output of the logical signal from the signal lines 606 and 608.

S412 corresponds to a transmission standby period. S414 corresponds to a transmission period (the transmission period includes a transmission preparation period in which a delay time calculation or the like is performed). The controller 112 outputs the transmission trigger signal to the relay module 200. In S412, in a case where the controller 206 in the relay module 200 receives the transmission trigger signal, the controller 206 starts outputting the transmission clock signal (first probe clock) to the transducer module 302. In S414, the transducer module 302 generates a delay amount set for the transmission beam forming based on the transmission clock signal, and then generates a plurality of transmission pulses based on the delay amount set. As a result, a transmission beam is formed.

At the end of S414, the level of the control signal on the signal line 608 is changed from High to Low. The level change means that the period in which the transmission is possible is ended, and the transition to the reception period, that is, the start of S416 is performed.

In S416, the reflected waves from the inside of the living body are received by the transducer array, which generates reception signal strings, and two reception signals in the reception signal strings are output from the transducer module 302 to the relay module 200 via the signal lines 604 and 606, and are output from the relay module 200 to the console.

S418 corresponds to S408 described above, and in S418, the dynamic parameter set is set in the same manner as described above. S408 (S418) to S416 are repeated until the user ends the examination or the like using the ultrasound, in other words, until the relay module 200 receives the end instruction from the console 100. In a case where the user gives an instruction to change the static parameter, such as a transmission frequency (for example, in order to obtain high resolution at a higher frequency or to ensure penetration at a lower frequency) or TGC (for example, in order to strengthen TGC since brightness at a short distance is high), the process returns to S404.

In the present embodiment, since the transmission beam forming can be executed using a clock with a high frequency, the captured image with high image quality can be displayed.

By the way, in the present embodiment, the register setting clock is set to a frequency lower than the frequency of the transmission clock. Since the transmission clock is used for the transmission beam forming, it is preferable that the frequency of the transmission clock is set to high. On the other hand, the register setting clock is a clock used in a case where the parameter is set in the register 322. In a case where the register setting clock has a high frequency, it may be possible to set the parameter in the register 322 in a short time. However, in a case where the circuit that transmits the register setting clock is made to be compatible with a high frequency clock, there is a possibility that the manufacturing cost and power consumption of the transducer module 302 may increase, and there is a problem of an increase in size of the electronic circuit.

Therefore, in the present embodiment, the frequency of the register setting clock is set to a frequency different from the frequency of the transmission clock, that is, a low frequency. The low frequency may be selected from a range of 10 to 40 MHz that does not correspond to the high frequency clock.

According to the present embodiment, even in a case where the console 100 and the relay module 200 are connected to each other by the long cable 4, the high frequency clock required for beam forming can be generated in the relay module 200, and the generated high frequency clock can be supplied from the relay module 200 to the probe 300 via the relatively short cable 6 (corresponding to a length of the catheter, generally 1 to 2 m). Therefore, a problem caused by long-distance transmission of the high frequency clock is less likely to occur. In addition, according to the configuration described above, since a plurality of types of clocks can be transmitted through a single signal line, there is an advantage that an increase in the thickness of the cable 8 (that is, the thickness of the probe 300) can be prevented or suppressed.

### Embodiment 2

In Embodiment 1 described above, a case where the high frequency clock is used for the transmission beam forming has been described. In Embodiment 2, the high frequency clock is also used for reception beam forming. Hereinafter, the case where the high frequency clock is used for the transmission beam forming will be described in detail.

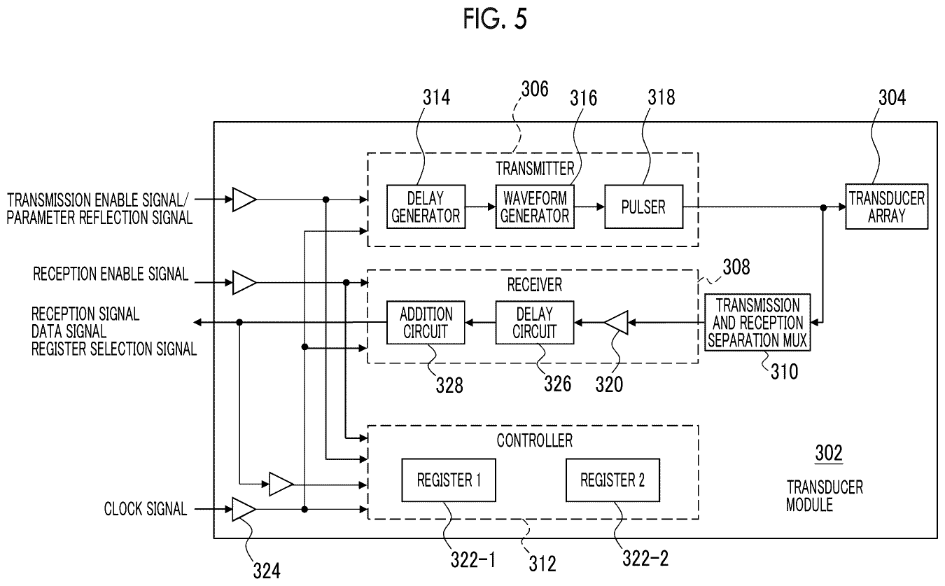

FIG. 5 is a diagram showing an example of a configuration of a body cavity insertion-type probe 300 in the present embodiment. In FIG. 5, the same components as the components included in the transducer module 302 of Embodiment 1 shown in FIG. 2 are denoted by the same reference numerals, and the description thereof will be omitted.

In the present embodiment, since the reception beam forming is executed, the internal configuration of the receiver 308 is different from the internal configuration of the receiver 308 according to Embodiment 1. In FIG. 5, the receiver 308 includes an amplifier 320, a delay circuit (delayer) 326, and an addition circuit 328. In practice, the receiver 308 includes a plurality of amplifiers 320, a plurality of delay circuits 326, and a plurality of addition circuits 328. For example, in a case where the transducer is configured with 12 reception openings (that is, in a case where the receiver 308 is configured with 12 reception channels), the receiver 308 is configured with 12 amplifiers 320, 12 delay circuits 326, and two addition circuits 328. It should be noted that one addition circuit 328 may be provided.

In the transmission and reception separation multiplexer 310, 12 reception signals corresponding to the 12 transducers constituting the reception opening are extracted in parallel from the reception signal strings output from the transducer array 304. The extracted 12 reception signals are input to 12 amplifiers 320. Each amplifier 320 amplifies the reception signal. The 12 delay circuits 326 apply the delay processing to the 12 amplified reception signals. Specifically, each delay circuit 326 applies the delay processing to each reception signal according to the reception delay time generated based on the clock. The two addition circuits 328 apply the addition processing to the 12 reception signals to which the delay processing is applied. Specifically, in each addition circuit 328, the six delayed reception signals are added, and thus an addition reception signal is generated. Two addition reception signals are generated from the two addition circuits 328. Each addition reception signal corresponds to the reception beam data.

Two reception signals (two addition reception signals) output from the receiver 308 are transmitted to the relay module 200 via two signal lines 604 and 606 as shown in FIG. 4.

In the present embodiment, the controller 312 also has a function of storing the parameter set for reception delay from the relay module 200 and generating the reception delay amount set based on the parameter set for reception delay.

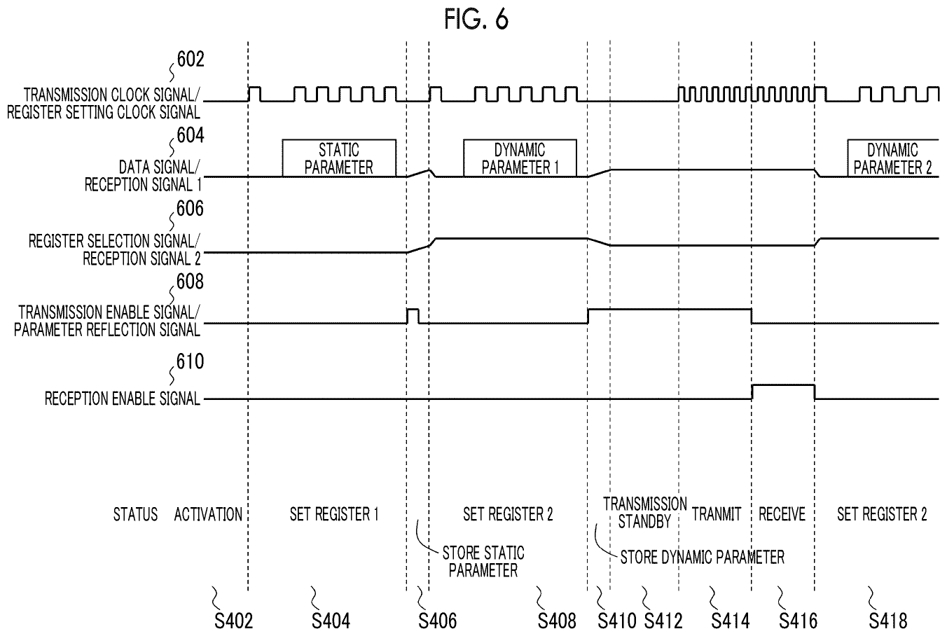

FIG. 6 is a timing chart of various signals exchanged between the relay module and the transducer module in a case where the reception beam forming is executed in the present embodiment. In the present embodiment, a signal line 610 is added to the signal lines 602 to 608 used in Embodiment 1.

In the present embodiment, the high frequency clock is also used in a case where the reception beam forming is executed. Accordingly, the relay module 200 outputs the high frequency clock to the transducer module 302 via the signal line 602 during the execution of the reception beam forming. In this case, since the level of the signal on the signal line 608 is Low, there is a possibility that the clock signal input from the signal line 602 is erroneously recognized as the register setting clock signal instead of the reception clock signal.

Therefore, in the present embodiment, a signal line 610 is added. The signal line 610 is a fifth signal line through which a reception enable signal is output from the relay module 200 to the transducer module 302. With reference to the reception enable signal, it is discriminated whether the clock signal input from the signal line 602 is a reception clock signal or a clock signal other than the reception clock signal.

In FIG. 6, it is assumed that the transmission clock signal used for the transmission beam forming is used as it is as the reception clock signal used for the reception beam forming. Therefore, in FIG. 6, the “transmission clock signal” is shown as it is.

Instead of providing the signal line 610, the clock signal input from the signal line 602 may be discriminated as the reception clock signal by the parameter. For example, a parameter that can specify a period for executing the reception beam forming, such as a time length or a counter, is stored in the register 2 as a dynamic parameter. Accordingly, the clock input from the signal line 602 while it can be determined that the reception period is being performed is referred to as a reception clock. The clock input from the signal line 602 after the end of the reception period is for register setting since the level of the signal of the signal line 608 is Low.

The relay module 200 relays the reception beam data output from the transducer module 302 to the console 100 by executing the reception beam forming.

In the present embodiment, since the reception beam forming can be executed using a clock with a high frequency, the captured image with high image quality can be displayed. In particular, according to the present embodiment, it is possible to acquire information on a plurality of reception signals, such as the reception opening 12ch, with only two signal lines 604 and 606. In the case of the reception opening 12ch, in Embodiment 1, the reception opening 12ch cannot be acquired unless transmission and reception are performed six times. However, in the present embodiment, the reception opening 12ch can be acquired by performing transmission and reception once. As a result, in the present embodiment, the image quality can be improved by increasing the frame rate or increasing the number of beams.

In addition, the configuration according to each of the above-described embodiments may be applied to an ultrasound diagnostic apparatus comprising a body cavity insertion-type probe that is inserted into a bronchus or the like.

What is claimed is:

1. A relay device comprising:
a first connection portion that is connected, via a first cable, to an ultrasound diagnostic apparatus main body having an original clock generation unit that generates an original clock;
a second connection portion that is connected, via a second cable, to a probe that is inserted into a body cavity; and
a probe clock generation unit that generates a probe clock used in the probe based on the original clock supplied from the ultrasound diagnostic apparatus main body via the first cable,
wherein the probe clock is supplied to the probe via the second cable.

2. The relay device according to claim 1,
wherein the probe clock generation unit generates, as the probe clock, a first probe clock used in generating a delay data set used for beam forming in the probe, and
a frequency of the first probe clock is higher than a frequency of the original clock.

3. The relay device according to claim 2,
wherein the probe clock generation unit further generates, as the probe clock, a second probe clock used in processing a parameter set used for the beam forming in the probe, and
a frequency of the second probe clock is different from the frequency of the first probe clock.

4. The relay device according to claim 3,
wherein the second cable has a signal line for a clock,
the first probe clock is output from the probe clock generation unit to the signal line for a clock in a first period, and
the second probe clock is output from the probe clock generation unit to the signal line for a clock in a second period different from the first period.

5. The relay device according to claim 4, further comprising:
a circuit that outputs, to the probe, a signal representing a type of the probe clock output to the signal line for a clock.

6. An ultrasound diagnostic apparatus comprising:
an ultrasound diagnostic apparatus main body having an original clock generation unit that generates an original clock;
a probe that is inserted into a body cavity; and
a relay device that is connected, via a first cable, to the ultrasound diagnostic apparatus main body and is connected, via a second cable, to the probe, the relay device including a probe clock generation unit that generates a probe clock used in the probe based on the original clock and outputting the probe clock to the probe via the second cable.

7. The ultrasound diagnostic apparatus according to claim 6,
wherein the probe clock generation unit selectively generates, as the probe clock, a first probe clock having a first frequency and a second probe clock having a second frequency lower than the first frequency,
the probe has an electronic circuit that executes beam forming, and
the electronic circuit
generates a delay data set used for the beam forming based on the first probe clock, and
stores a parameter set used for the beam forming, which is a parameter set transmitted from the relay device, based on the second probe clock.

8. The ultrasound diagnostic apparatus according to claim 7,
wherein the relay device
outputs the first probe clock and the second probe clock to the probe via a signal line for a clock in the second cable, and
outputs a signal representing a type of the probe clock to the probe via the second cable, and
the electronic circuit identifies a type of the probe clock input via the signal line for a clock according to the signal representing the type.

9. The ultrasound diagnostic apparatus according to claim 8,
wherein the parameter set includes a first parameter set that is common over a plurality of times of the beam forming and a second parameter set that is changeable for each beam forming,
the relay device
outputs the first parameter set and the second parameter set as the parameter set to the probe via the second cable, and
outputs a signal representing a storage location of the parameter set to the probe, and
the electronic circuit stores the first parameter set in a first storage location and stores the second parameter set in a second storage location based on the signal representing the storage location of the parameter set.

10. The ultrasound diagnostic apparatus according to claim 9,
wherein the second cable has a first signal line, a second signal line, a third signal line, and a fourth signal line,
the first signal line is the signal line for a clock,
the second signal line is a signal line for transmitting the parameter set from the relay device to the electronic circuit and for transmitting a first reception signal from the electronic circuit to the relay device,
the third signal line is a signal line for transmitting the signal representing the storage location of the parameter set from the relay device to the electronic circuit and for transmitting a second reception signal from the electronic circuit to the relay device, and
the fourth signal line is a signal line for transmitting the signal representing the type of the probe clock from the relay device to the electronic circuit.
